(19) United States
(12) Patent Application Publication
(10) Pub. No.: US 2023/0255985 A1
(43) Pub. Date: Aug. 17, 2023

(54) METHODS AND COMPOSITIONS FOR TREATMENT OF HEAD LICE

(71) Applicant: CReDO Science, LLC, Austin, TX (US)

(72) Inventor: Ethan Russo, Vashon, WA (US)

(21) Appl. No.: 18/000,586

(22) PCT Filed: Jun. 9, 2021
(86) PCT No.: PCT/US2021/036685

§ 371 Date: Dec. 2, 2022

Related U.S. Application Data
(60) Provisional application No. 63/036,842, filed on Jun. 9, 2020.

(51) Int. Cl. A61K 31/00; A61K 31/015; A61K 47/44; A61P 33/14; A61K 9/00

(52) U.S. Cl. CPC A61K 31/658; A61K 9/0014; A61K 31/015; A61K 47/44; A61P 33/14

(57) ABSTRACT

The current invention relates to compositions and methods for treating head lice using compounds extracted from Cannabis sativa. The method can include a formulation including a compound extracted from cannabis and coconut oil.

### CLAIM OF PRIORITY UNDER 35 U.S.C. §119

The present Application for Patent claims priority to Provisional Application No. 63/036,842 entitled “METHODS AND COMPOSITIONS FOR TREATMENT OF HEAD LICE” filed Jun. 9, 2020 and assigned to the assignee hereof and hereby expressly incorporated by reference herein.

### BACKGROUND

### Field

The current invention relates to compositions, methods, and kits for removing, treating or preventing head lice using compounds extracted from Cannabis. The method can include a formulation including compounds extracted from Cannabis and coconut oil.

### Background

Head lice have become a major public health nuisance in the United Kingdom, with recent estimates that 25% of school-age children are affected each year. In the United States, 6-12 million people are infested each year with head lice (Centers for Disease Control 2001). Current treatment is mechanical or chemical. Mechanical treatment utilizes a fine-toothed comb to remove head lice and eggs (“nits”). Wet combing must be repeated every 2-3 days over a 2-week period. Compliance with this regimen is often poor, and a randomized trial showed that the cure rate using wet combing was 38%, compared to a cure rate of 78% using chemical (insecticide) treatment (Roberts et al. 2000). Four chemicals are commonly available in the United States for treating head lice but are rarely tested in children ≤ 2 years old due to fears of attendant toxicity.

### SUMMARY

Some embodiments of the invention relate to a formulation including a combination of at least two active substances extracted from Cannabis and a carrier oil, wherein the formulation can be effective in controlling head lice by disruption of at least one phase of a louse life cycle. In some embodiments, the two active substances have a synergistic effect on lice control.

In some embodiments, at least one of the active substances is a cannabinoid, a terpene, or a flavonoid.

In some embodimnets, the active substances can be selected from CBGA, CBDA, CBCA, THCA, linalool, nerolidol, limonene, alpha-pinene, guaiol, caryophyllene, caryophyllene oxide, beta-eudesmol, phellandrene, eugenol, and/or the like.

In some embodiments, the formulation can include CBGA and linalool.

In some embodiments, the formulation can include CBDA and linalool.

In some embodiments, the formulation can include CBCA and linalool.

In some embodiments, the formulation including CBGA and linalool, CBDA and linalool, or CBCA and linalool can further include nerolidol, limonene and alpha-pinene.

In some embodiments, the formulation can include CBGA and nerolidol.

In some embodiments, the formulation can include CBDA and nerolidol.

In some embodiments, the formulation can include CBGA, CBDA, CBCA, THCA, linalool, nerolidol, limonene, alpha-pinene, guaiol, caryophyllene, caryophyllene oxide, beta-eudesmol, phellandrene, and eugenol.

In some embodiments, the formulation can include THCA, linalool, nerolidol, limonene, and alpha-pinene.

In some embodiments, the carrier oil is coconut oil or the like.

In some embodiments, the formulation can include at least three active ingredients.

In some embodiments, all active ingredients are cannabinoids.

In some embodiments, all active ingredients are terpenes.

In some embodiments, the formulation can include a combination of: (a) at least one cannabinoid with at least one terpene; or (b) at least one cannabinoid with at least one flavonoid; or (c) at least one terpene with at least one flavonoid; or (d) at least one cannabinoid, at least one terpene, and at least one flavonoid.

In some embodiments, the formulation can include at least one emulsifier.

Some embodiments of the invention relate to a method for treatment of head lice in a patient. The method can include applying the formulation provided herein to the scalp of the patient; removing the formulation after a period of time; and combing the hair of the patient to remove the head lice.

### BRIEF DESCRIPTION OF THE DRAWINGS

FIG. 1 is a table of pairwise combinations of synergistic ingredients of the invention wherein the ingredients are cannabinoids, flavonoids, or a cannabinoid and a flavonoid.

FIG. 2 is a table of pairwise combinations of synergistic ingredients of the invention wherein the ingredients are terpenes.

FIG. 3 is a table of pairwise combinations of synergistic ingredients of the invention wherein the ingredients.

FIG. 4 is a table listing major ratios of pairwise combinations of synergistic ingredients of the invention, named as ratios A through S.

FIG. 5 lists additional synergistic ingredients that are added to the compositions in certain embodiments.

### DETAILED DESCRIPTION

Compositions for controlling lice or formulations including the composition including at least two active compounds derived from a Cannabis plant are provided.

The compositions and methods of the invention can be used to control any type of arthropod, such as an insect. Exemplary arthropods that can be controlled include but are not limited to beetles, cockroaches, flies, ants, larvae, bees, lice, fleas, mosquitoes, moths, and the like. Exemplary arthropod orders can include but are not limited to orders Acari, Anoplura, Araneae, Blattodea, Coleoptera, Collembola, Diptera, Grylloptera, Hemiptera, Heteroptera, Homoptera, Hymenoptera, Isopoda, Isoptera, Lepidoptera, Mantodea, Mallophaga, Neuroptera, Odonata, Orthoptera, Psocoptera, Siphonaptera, Symphyla, Thysanura, and Thysanoptera and the like.

The compositions and methods of the invention can used to control any type of lice (order Phthiraptera) including Anoplura (sucking lice) and Mallophaga (chewing lice). Exemplary suborders of lice that can be controlled include but are not limited to, Anoplura (occurring on mammals); Rhyncophthirina (parasites of elephants and warthogs); Ischnocera (mostly avian chewing lice); and Amblycera (a primitive suborder of chewing lice, widespread on birds, however, also live on South-American and Australian mammals) and the like.

For purposes of simplicity, the term “lice” is used in this application, however the invention can be applied to any arthropod. However, in some embodiments, the term “lice” applies to lice as described above. Also for purposes of this application, the term “control” refers to having a repellent effect, a pesticidal effect, ovicidal effect, or the like, or any combination thereof.

“Repellent effect” is an effect wherein more lice are repelled away from a host or area that has been treated with the composition than a control host or area that has not been treated with the composition. In some embodiments, repellent effect is an effect wherein at least about 50% of lice are repelled away from a host or area that has been treated with the composition. In some embodiments, repellent effect is an effect wherein at least about 75% of lice are repelled away from a host or area that has been treated with the composition. In some embodiments, repellent effect is an effect wherein at least about 90% of lice are repelled away from a host or area that has been treated with the composition. In some embodiments, repellent effect is an effect wherein at least about 95% of lice are repelled away from a host or area that has been treated with the composition.

“Pesticidal effect” is an effect wherein treatment with a composition causes at least about 1% of the lice to die. In this regard, an LC1 to LC100 (lethal concentration) or an LD1 to LD100 (lethal dose) of a composition will cause a pesticidal effect. In some embodiments, the pesticidal effect is an effect wherein treatment with a composition causes at least about 5% of the exposed lice to die.

In some embodiments, the pesticidal effect is an effect wherein treatment with a composition causes at least about 10% of the exposed lice to die. In some embodiments, the pesticidal effect is an effect wherein treatment with a composition causes at least about 25% of the lice to die. In some embodiments the pesticidal effect is an effect wherein treatment with a composition causes at least about 50% of the exposed lice to die. In some embodiments the pesticidal effect is an effect wherein treatment with a composition causes at least about 75% of the exposed lice to die. In some embodiments the pesticidal effect is an effect wherein treatment with a composition causes at least about 90% of the exposed lice to die.

In some embodiments of the invention, treatment with compositions of the invention will result in a knockdown of the lice occurring within a few seconds to a few minutes. “Knockdown” is an effect wherein treatment with a composition causes at least about 1% of the exposed lice to display reduced mobility. In some embodiments, the knockdown is an effect wherein treatment with a composition causes at least about 50% of the exposed lice to display reduced mobility. In some embodiments, the treatment with the composition causes at least about 50%, 55%, 60%, 65%, 70%, 75% or more of the exposed live to display reduced mobility.

Embodiments of the invention are directed to compositions for controlling lice and methods for using these compositions. In some embodiments, the compositions can include compounds that are generally regarded as safe (GRAS compounds). In some embodiments, the compositions can include compounds derived from a Cannabis plant, including, but not limited to, cannabinoids or terpenoids. In some embodiments, the compositions include two or more compounds that act synergistically.

The present invention comprises compositions for controlling lice, which can include two or more active compounds derived from Cannabis and methods for using these compositions. Each active compound can make up between about 0.1% to about 99%, by weight, of the composition mixture. The Cannabis compounds, when combined, can have a synergistic effect. In some embodiments, the composition can include at least three, four, or five or more active compounds, in any effective combination. The compositions of the present invention can include any of the following Cannabis compounds listed below, or mixtures thereof:

TABLE 1

Exemplary Cannabinoids

| | |
|---|---|
| THC (tetrahydrocannabinol) | THCA (tetrahydrocannabinolic acid) |
| CBD (cannabidiol) | CBDA (cannabidiolic acid) |
| CBN (cannabinol) | CBG (cannabigerol) |
| CBC (cannabichromene) | CBL (cannabicyclol) |
| CBV (cannabivarin) | THCV (tetrahydrocannabivarin) |
| CBDV (cannabidivarin) | CBCV (cannabichromevarin) |
| CBGV (cannabigerovarin) | CBGM (cannabigerol monomethyl ether) |
| CBE (cannabielsoin) | CBT (cannabicitran) |
| CBCA (cannabichromenic acid) | CBGA (cannabigerolic acid) |

TABLE 2

Exemplary Terpenes

| | | | |
|---|---|---|---|
| 3-Carene | Alpha Pinene | Beta Caryophyllene | Beta Pinene |
| Bisabolol | Borneol | Camphene | Camphor |
| Caryophyllene | Caryophyllene Oxide | Cedrene | Cedrol |
| Delta 3 Carene | Eucalyptol | Fenchol | Fenchone |
| Geraniol | Geranyl Acetate | Guaiol | Humulene |
| Isoborneol | Isopulegol | Limonene | Linalool |
| Menthol | Myrcene | Nerol | Nerolidol |
| Ocimene | Phellandrene | Phytol | Pulegone |
| Sabinene | Terpinene | Terpineol | Terpinolene |
| Valencene | Caryophyllene oxide | Beta-eudesmol | Eugenol |

TABLE 3

Exemplary Flavonoids

| | | | |
|---|---|---|---|
| Cannflavin A | Cannflavin B | Cannflavin C | Orientin |
| Quercetin | Silymarin | Kaempferol | |

“Compound derived from a Cannabis plant,” as used herein, can be defined as a compound naturally found in Cannabis. The actual compound used in the composition that is naturally found in Cannabis can be produced from another source.

In some embodiments, the composition can include THCA, CBGA, CBDA, CBG, and/or CBD. In some embodiments, the composition can include an acid cannabinoid. In some embodiments, the composition does not include THC. In some embodiments, the composition can include limonene, pinene, caryophyllene oxide, guaiol, terpineol, terpinene, nerolidol and/or myrcene.

In some embodiments, the composition or formulation including the composition can include about 1%, 5%, 10%, 15%, 20%, 25%, 30%, 35%, 40%, 45%, 50%, 55%, 60%, 65%, 70%, 75%, 80%, 85%, 90% or more cannabinoid by weight.

In some embodiments, the composition or formulation including the composition can include about 0.001%, 0.01%, 0.1%, 1%, 5%, 10%, 15%, 20%, 25%, 30%, 35%, 40%, 45%, 50%, 55%, 60%, 65%, 70%, or more terpenoid or terpene by weight.

The two compounds together can have synergistic effects of the control of head lice as provided in the Figures. For example, formula 1 includes a combination of THC and THCA which exhibits synergistic effects. For example, formula 1933 includes a combination of 3-Carene and THC which exhibits synergistic effects.

The use of the terms, “synergistic” and “synergistically effective,” are used in the present invention to mean a biological effect created from the application of two or more agents to produce a biological effect that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

While embodiments of the invention can include active ingredients, carriers, inert ingredients, and other formulation components, preferred embodiments begin with a primary blend. A primary blend is preferably a synergistic combination containing two or more active ingredients and, optionally, additional ingredients. The primary blends can then be combined with other ingredients to produce a formulation. Accordingly, where concentrations, concentration ranges, or amounts, are given herein, such quantities typically are in reference to a primary blend or blends. Thus, when a primary blend is further modified by addition of other ingredients to produce a formulation, the concentrations of the active ingredients are reduced proportional to the presence of other ingredients in the formulation.

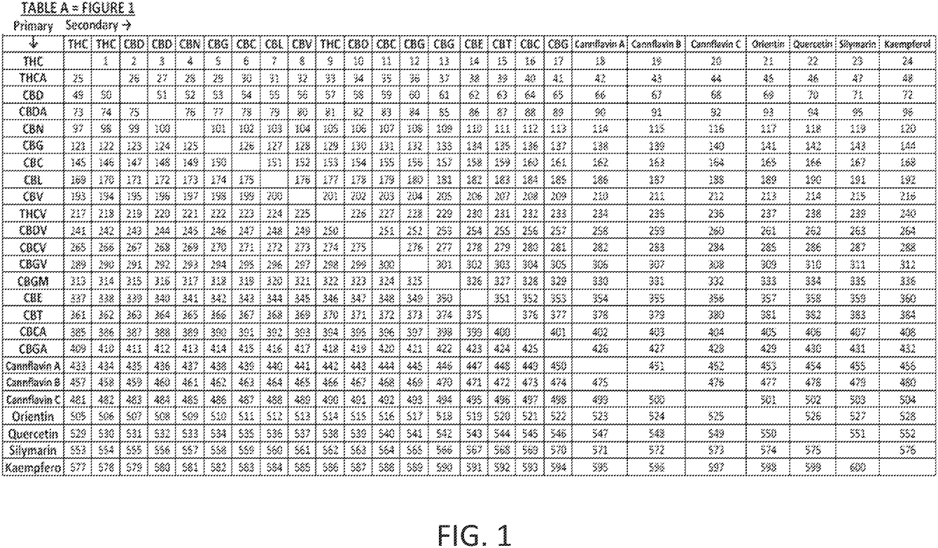

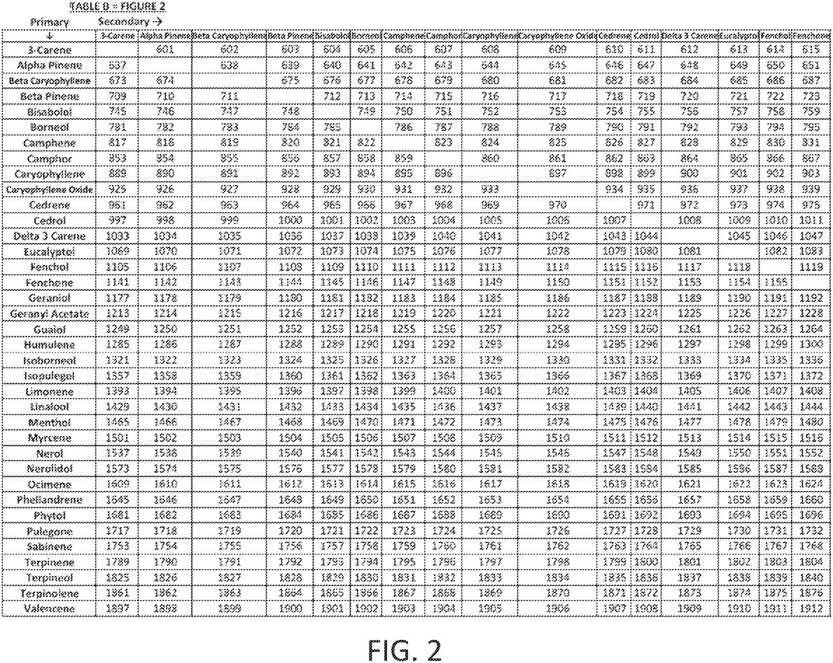

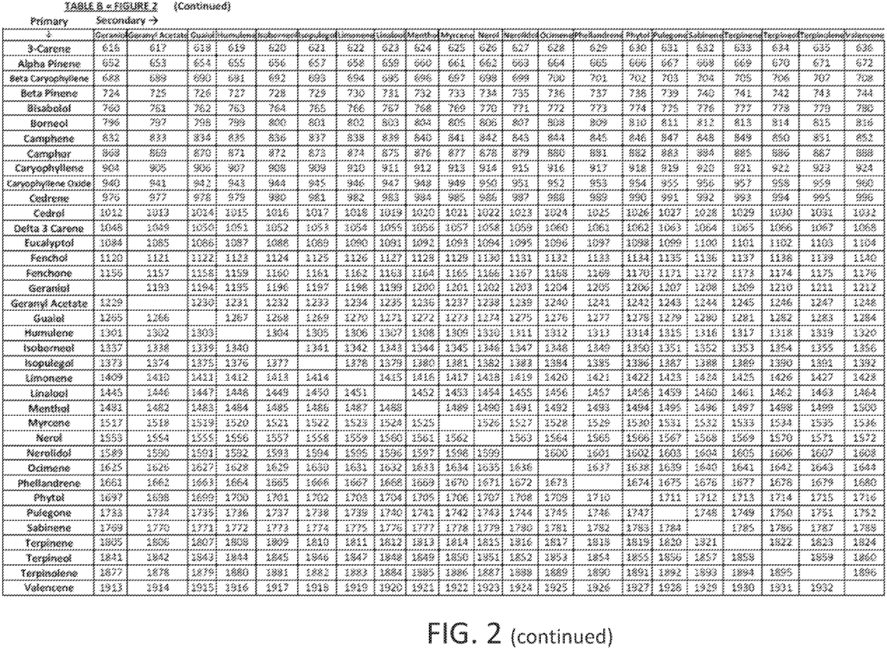

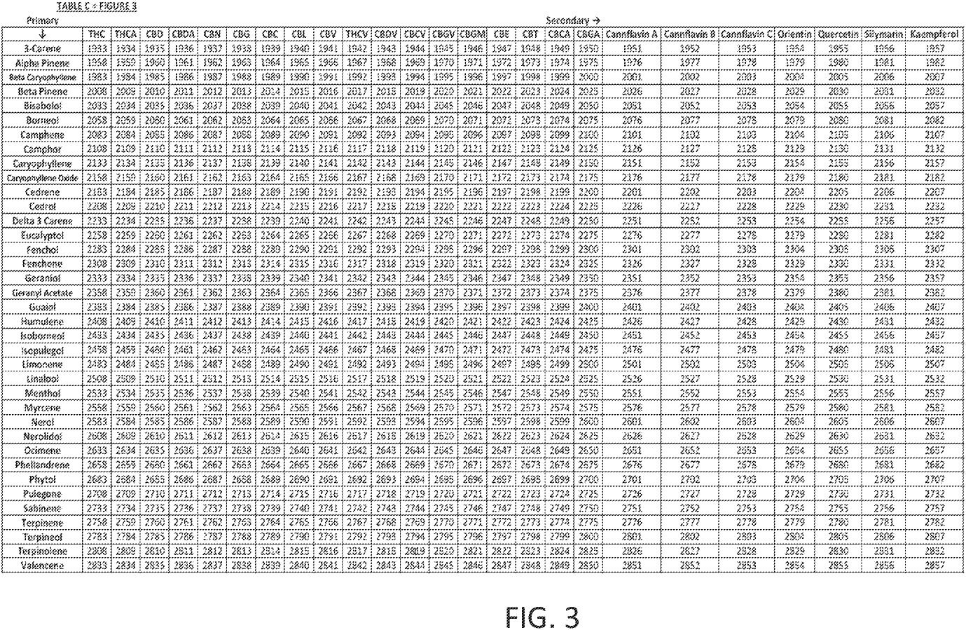

FIGS. 1-3 provide tables of pairwise combinations of synergistic ingredients of the invention wherein the ingredients. FIG. 1 provides combinations wherein the synergistic ingredients are cannabinoids, flavonoids, or a cannabinoid and a flavonoid. FIG. 2 provides combinations wherein the synergistic ingredients are terpenes. FIG. 3 provides combinations wherein the synergistic ingredients are a terpene and a cannabinoid or a terpene and a flavonoid. For example, in some embodiments, the synergistic ingredients are CBGA and linalool (Formula 2525). In other embodiments, the synergistic ingredients are CBDA and linalool (Formula 2511). In other embodiments, the synergistic ingredients are CBCA plus linalool (Formula 2524). In other embodiments, the synergistic ingredients are CBGA and nerolidol (Formula 2625). In other embodiments, the synergistic ingredients are CBDA and nerolidol (Formula 2611).

The ratio of the synergistic ingredients can be about 1:1, 1:2, 1:3, 1:6, 1:9, 1:12, 2:1, 2:3, 2:9, 3:1, 3:2, 3:12, 6:1, 9:1, 9:2, 9:12, 12:1, 12:3, 12:9 or more or less.

For example, the formulation can exist in a range of concentration ratios such as 3:1, 1:1, or 1:3. Likewise, depending upon the stage of the louse life cycle targeted and, in some cases, the species of louse targeted, the optimized formula (as described in Examples 2-4) can be 12 (or more):1, 9:1, 3:1, 2:1, 1.5:1, 1:1, 1:1.5, 1:2, 1:3, 1:6, 1:9, 1:12 (or more), or the like.

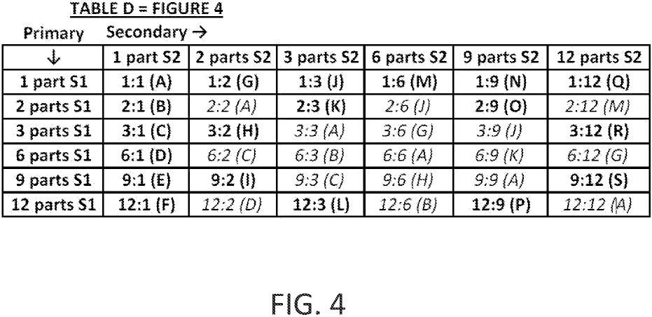

FIG. 4 lists major ratios of pairwise combinations of synergistic ingredients of the invention, named as ratios A through S. While the table in this figure provides a range of ratios that can be useful, it is within the scope of the invention to provide formulations in which synergistic ratios are adapted to particular uses.

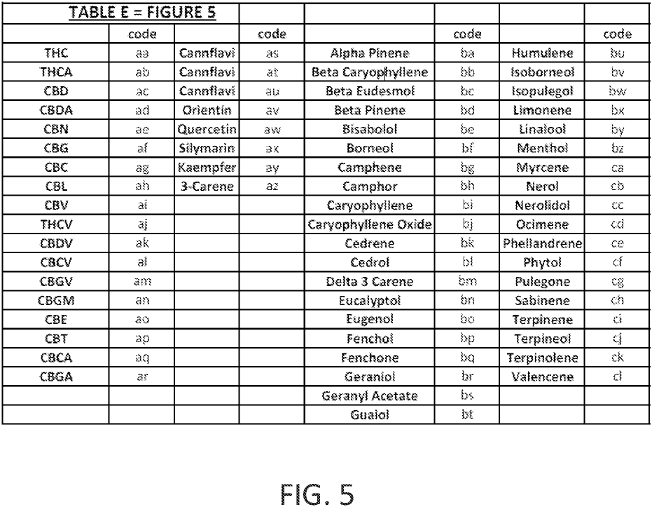

Likewise, the synergistic formulations of the invention can include additional ingredients to further synergistically enhance the efficacy of a pairwise combination. In such embodiments, a paired combination is enhanced with a tertiary ingredient selected from FIG. 5. In some embodiments, a quaternary ingredient can also be selected from FIG. 5. Likewise in other embodiments, additional synergists can be selected, either from FIG. 5 or from other sources based upon desired effects.

The compositions and formulations of the present invention can comprise one or more suitable carriers. A suitable carrier can include any carrier in the art known for Cannabis compounds, provided the carrier does not adversely affect the compositions of the present invention. The term “carrier” as used herein means an inert or fluid material, which can be inorganic or organic and of synthetic or natural origin, with which the active compound is mixed or formulated to facilitate its application to the host, area, or other object to be treated, or to facilitate its storage, transport and/or handling. In general, any of the materials customarily employed in formulating repellents, pesticides, herbicides, or fungicides, are suitable.

In preferred embodiments the actives are dissolved or suspended in one or more carrier oils such as, for example, coconut oil. A non-limiting list of exemplary carrier oils suitable for topical application is provided in Table 5.

TABLE 5

Exemplary Carrier Oils

| | | |
|---|---|---|
| Jojoba oil | Apricot kernel oil | Coconut oil |
| Sweet almond oil | Olive oil | Argan oil |
| Rosehip oil | Black seed oil | Grape seed oil |
| Avocado oil | Sunflower oil | Shea butter |
| Palm oil | | |

The compositions of the present invention can be employed alone or in the form of mixtures with such solid and/or liquid dispersible carrier vehicles and/or other known compatible active agents such as other repellents, pesticides, or acaricides, nematicides, fungicides, bactericides, rodenticides, herbicides, fertilizers, growth-regulating agents, etc., if desired, or in the form of particular dosage preparations for specific application made therefrom, such as solutions, emulsions, suspensions, powders, pastes, and granules which are thus ready for use.

The compositions of the present invention can be formulated or mixed with, if desired, conventional inert pesticide diluents or extenders of the type usable in conventional lice control agents, e.g., conventional dispersible carrier vehicles such as gases, solutions, emulsions, suspensions, emulsifiable concentrates, spray powders, pastes, soluble powders, wettable powders, dusting agents, granules, foams, mousses, pastes, tablets, aerosols, amorphous silica, natural and synthetic materials impregnated with active compounds, microcapsules, etc.

Likewise, in some compositions, an optional emulsifier such as, for example lecithin, can be used to modify the texture and consistency of the composition to make it easier to apply and/or remove. A non-limiting list of exemplary emulsifiers considered safe and suitable for topical applications is provided in Table 6.

TABLE 6

Exemplary Natural Emulsifiers

| | | |
|---|---|---|
| Cetearyl Alcohol | Cetearyl Glucoside | Cetearyl Olivate |
| Cetearyl wheat straw glycosides | Glycerol Stearate | Polyglyceryl-2 Stearate |
| Sorbitan Olivate | Stearyl Alcohol | Sucrose Stearate |

The compositions of the present invention can be used to control lice by treating a host directly. For example, the host can be treated directly by using a cream or spray formulation, which can be applied externally or topically, e.g., to the skin or hair of a human. A composition can be applied to the host, for example, in the case of a human, using formulations of a variety of personal products or cosmetics for use on the skin or hair. For example, any of the following can be used: fragrances, colorants, pigments, dyes, colognes, skin creams, skin lotions, deodorants, talcs, bath oils, soaps, shampoos, hair conditioners, mousses, and styling agents.

In certain embodiments, the lice control composition is provided as a lotion, cream, spray, shampoo, foam, mousse, and/or the like.

In some embodiments, the composition includes two compounds derived from a Cannabis plant and coconut oil. In some embodiments, another ingredient with similar properties to coconut oil can be used as a substitute, or in addition to the coconut oil. In some embodiments, the coconut oil (or substitute) and the compound(s) extracted from Cannabis have synergistic properties.

In some embodiments, compounds derived from Cannabis can be a terpenoid, a cannabinoid, or any other compound derived from Cannabis or any combination thereof. For example, the terpenoid can be one or more of limonene, pinene, terpinene, terpineol, terpinen-4-ol, nerolidol, eugenol, myrcene and/or the like, and/or combinations of the same. For example, the cannabinoid can be THCA, CBGA, CBDA, CBCA, CBG, CBD and/or at least one other cannabinoid. For example, the compound extracted from Cannabis can be a methyl ketone, caryophyllene oxide and/or guaiol, and/or the like.

In some embodiments, the formulation can include one or more cannabinoid in the range of 0.1-25% total cannabinoid by weight. In some embodiments, the formulation can include one or more terpenoid in the range of 0.1-15% total terpenoid. In some embodiments, the formulation includes one or more cannabinoids in the range of 1-10%. For example, the cannabinoid can be in the concentration of about 1, 2, 3, 4, 5, 6, 7, 8, 9, 10, or more percent of the formulation. In some embodiments, the formulation includes one or more terpenoids in the range of 0.5-5%. For example, the cannabinoid can be in the concentration of about 0.5, 1, 1.5, 2, 2.5, 3, 3.5, 4, 4.5, 5, 6, 7, 8, 9, 10 or more percent of the formulation. In some embodiments, the two compounds have synergistic properties.

In some embodiments, the formulation can further include compounds not found in Cannabis, such as the terpenoid thymol or other terpenoids that display octopaminergic agonism or other insecticidal properties. In some embodiments, the compound(s) extracted from cannabis and the compounds not in cannabis have synergistic properties.

### Methods

Some embodiments of the invention relate to a method of treating a patient with head lice with the formulation. The method can include applying the formulation to hair/scalp of the patient. The pediculicidal formulation of phytocannabinoids and terpenoids in coconut oil base can be applied to the hair for a period of time. The period of time can be at least about 0.5, 1, 2, 3, 4, 5, 6, 7, 8, 9, or 10 hours or more. For example, the formulation can be applied with gloves before bedtime, and can melt at body temperature as it is massaged into the scalp. For example, in some embodiments the following steps can be used:
The hair can be covered with a hairnet or shower cap (for example, before sleep.)
After the period of time (for example in the morning), the formulation can be shampooed from the scalp.
This can be followed by combing to remove lice and nits.
Clothing and bed linens can be laundered in hot water.
The process can be repeated in two weeks.

### Kits

Some embodiments of the invention relate to kits to carry out any of the methods disclosed herein. The kit can include the composition, means of applying the composition, and/or a comb (or similar device to remove lice). In some embodiments, an active ingredient fraction is provided in one container. The fraction in the container is to be added at the time of application to the carrier oil, such as coconut oil.

### EXAMPLES

### Example 1 - Establishing and Quantifying Synergistic Combinations

Formulations of the present invention are prepared based upon the methodology previously pursued by Priestley, C. M., I. F. Burgess, and E. M. Williamson. 2006. “Lethality of essential oil constituents towards the human louse, Pediculus humanus, and its eggs.” Fitoterapia 77 (4): 303-9. https://doi.org/10.1016/j.fitote.2006.04.005. http://www.ncbi.nlm.nih.gov/pubmed/16707234, which is incorporated by reference in its entirety herein, and based on previous World Health Organization guidelines (World Health Organisation. 1981. Instructions for determining the susceptibility or resistance of body lice and head lice to insecticides.https://apps.who.int/iris/handle/10665/70733, which is incorporated by reference in its entirety herein). Combinations of two or more of CBGA, CBDA, CBCA, THCA plus linalool, nerolidol, limonene, alpha-pinene, guaiol, caryophyllene, caryophyllene oxide, beta-eudesmol, phellandrene, eugenol. Each combination is diluted to 1% and 10% solutions in a suitable diluent for the assays to assess effects on live lice and louse eggs. Control tests are done with the diluent alone and with a positive control (carbaryl et al.) in a double-blind fashion to confirm and quantify the relative pediculicidal benefits of the various compositions. Effective combinations are observed based upon killing of live lice and failure of louse eggs (“nits”) to hatch. Synergies of the compositions are quantified at different concentrations of each ingredient, as compared with the activity of each ingredient alone, where an adaptation of the Colby method of synergy quantification is employed. See S. R. Colby, “Calculating Synergistic and Antagonistic Response Of Herbicide Combinations”, Weeds, 15(1): 20-23, 1967.Several combinations demonstrate synergistic effects.

### Example 2 - Optimizing Synergistic Combinations

Pairwise combinations of ingredients as described in Example 1 showing activity against one of more stages of the louse life cycle are selected for ratio optimization, in 3:1, 1:1, and 1:3 ratios and re-tested for effectiveness against each stage of the life cycle. The most effective concentrations against each phase of the life cycle are further optimized based upon the results of the initial ratio optimization wherein, for combinations showing best results in 3:1 ratios, additional activity assays are conducted at ratios of 6:1 and 9:1; likewise, for combinations showing best results at 1:3, additional activity assays are conducted at ratios of 1:6 and 1:9. For combinations showing best results at 1:1, additional activity assays are conducted at ratios of 2;1, 1.5:1, 1:1.5, and 1:2.

### Example 3

Combinations from Examples 2 and 3 are tested empirically by addition of a third, fourth, and fifth active ingredient in ratios as set forth in Example 2, then the most strongly effective and/or synergistic compositions are further tested for optimization as in Example 2.

### Example 4

Effective compositions from any of Examples 1-3 are modified by employing a variety of carrier oils, and tested for disruption of the louse life cycle as described above. One or more optimal carrier oils for each synergistic combination are determined.

### Example 5

Formulations from any of Examples 1-4 are combined with one or more emulsifiers and/or other suitable modifiers of texture or other properties to develop products most suitable for use in hair as well as to develop products most amenable to repeated use with minimal inconvenience and discomfort.

### Example 6

Referring to the Figures, Formula 2525 + cc + bx + ba is prepared. The formulation of CBGA, linalool, nerolidol, limonene and alpha-pinene is found to be effective in the treatment of head lice.

### Example 7

Referring to the Figures, Formula 2511 + cc + bx + ba is prepared. The formulation of CBDA, linalool, nerolidol, limonene, and alpha-pinene is prepared and found to be effective in the treatment of head lice.

### Example 8

Referring to the Figures, Formula 2524 + cc + bx + ba is prepared. The formulation of CBCA, linalool, nerolidol, limonene, and alpha-pinene is found to be effective in the treatment of head lice.

### Example 9

Referring to the Figures, Formula 2509 + cc + bx + ba is prepared. The formulation of THCA, linalool, nerolidol, limonene, alpha-pinene is found to be effective in the treatment of head lice.

### Example 10

Referring to the Figures, Formula 2625 + ad + aq + ab + by + bx + ba + bt + bi + bj + be + ce + bo is prepared. The formulation of CBGA, CBDA, CBCA, THCA plus linalool, nerolidol, limonene, alpha-pinene, guaiol, caryophyllene, caryophyllene oxide, beta-eudesmol, phellandrene, eugenol is prepared and found to be effective in the treatment of head lice.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A formulation comprising a combination of at least two active substances extracted from Cannabis and a carrier oil, wherein the formulation is effective in controlling head lice by disruption of at least one phase of a louse life cycle, wherein the two active substances have a synergistic effect on lice control.

2. The formulation of claim 1, wherein at least one of the active substances is a cannabinoid, a terpene, or a flavonoid.

3. The formulation of claim 1, wherein the active substances are selected from CBGA, CBDA, CBCA, THCA, linalool, nerolidol, limonene, alpha-pinene, guaiol, caryophyllene, caryophyllene oxide, beta-eudesmol, phellandrene, and eugenol.

4. The formulation of claim 3, comprising CBGA and linalool.

5. The formulation of claim 3, comprising CBDA and linalool.

6. The formulation of claim 3 comprising CBCA and linalool.

7. The formulation of any of claims 4-6 further comprising nerolidol, limonene and alpha-pinene.

8. The formulation of claim 3 comprising CBGA and nerolidol.

9. The formulation of claim 3 comprising CBDA and nerolidol.

10. The formulation of claim 3, comprising CBGA, CBDA, CBCA, THCA, linalool, nerolidol, limonene, alpha-pinene, guaiol, caryophyllene, caryophyllene oxide, beta-eudesmol, phellandrene, and eugenol.

11. The formulation of claim 3, comprising THCA, linalool, nerolidol, limonene, and alpha-pinene.

12. The formulation of claim 1, wherein the carrier oil is coconut oil.

13. The formulation of claim 1, comprising at least three active ingredients.

14. The formulation of claim 1, wherein all active ingredients are cannabinoids.

15. The formulation of claim 1, wherein all active ingredients are terpenes.

16. The formulation of claim 1, comprising a combination of:
a. at least one cannabinoid with at least one terpene; or
b. at least one cannabinoid with at least one flavonoid; or
c. at least one terpene with at least one flavonoid; or
d. at least one cannabinoid, at least one terpene, and at least one flavonoid.

17. The formulation of claim 1, further comprising at least one emulsifier.

18. A method for treatment of head lice in a patient comprising
applying the formulation of claim 1 to the scalp of the patient;
removing the formulation after a period of time; and
combing the hair of the patient to remove the head lice.
